(19) United States
(12) Patent Application Publication
(10) Pub. No.: US 2025/0194921 A1
(43) Pub. Date: Jun. 19, 2025

(54) MEDICAL OBSERVATION DEVICE AND INFORMATION PROCESSING DEVICE

(71) Applicant: SONY GROUP CORPORATION, TOKYO (JP)

(72) Inventors: YUGO KATSUKI, TOKYO (JP); TAKASHI YAMAGUCHI, TOKYO (JP); TOMOYUKI OOTSUKI, TOKYO (JP)

(21) Appl. No.: 18/849,316

(22) PCT Filed: Mar. 20, 2023
(86) PCT No.: PCT/JP2023/010801

§ 371 Date: Sep. 20, 2024

(30) Foreign Application Priority Data
Mar. 29, 2022 (JP) 2022-054384

(51) Int. Cl. A61B 3/13; A61B 3/12

(52) U.S. Cl. CPC A61B 3/13; A61B 3/12; A61B 3/1225

(57) ABSTRACT

A medical observation device includes: a light source that emits at least spatially incoherent light; an image sensor that acquires an image of the light emitted from the light source and reflected by a subject; and a signal processing unit that corrects a signal level obtained from first image data acquired by the image sensor, based on a wavefront aberration of the light reflected by the subject.

### FIELD

The present disclosure relates to a medical observation device and an information processing device.

### BACKGROUND

In a medical field such as ophthalmic examination and surgery, for example, there has been an actively utilized technology referred to as optical coherence tomography (OCT) technology that uses optical coherence to capture an image of an internal structure of an object (for example, eyeball) with high resolution and high speed. Furthermore, in recent years, a full-field optical coherence tomography (FFOCT) technology has been developed to concurrently acquire two-dimensional images on the XY plane by using a two-dimensional imaging device as a light receiver.

### CITATION LIST

### Patent Literature

Patent Literature 1: WO 2014/192520 A

### SUMMARY

### Technical Problem

Conventional OCT in general applications uses a light source that emits coherent light (hereinafter, also referred to as a coherent light source), with the coherent light maintaining a constant and temporally invariable phase relationship between light waves at optionally determined two points in a light flux. Therefore, in a case of observing a subject that might have aberration, such as a human eye, for example, there is a possibility that image quality (for example, resolution) of an observed image is degraded due to the influence of the aberration.

In view of this, the present disclosure proposes a medical observation device and an information processing device capable of suppressing degradation in image quality.

### Solution to Problem

A medical observation device according to an embodiment of the present disclosure includes: a light source that emits at least spatially incoherent light; an image sensor that acquires an image of the light emitted from the light source and reflected by a subject; and a signal processing unit that corrects a signal level obtained from first image data acquired by the image sensor, based on a wavefront aberration of the light reflected by the subject.

### BRIEF DESCRIPTION OF DRAWINGS

FIG. 1 is a schematic diagram illustrating a schematic configuration example of a medical observation device according to a first embodiment.

FIG. 2 is a diagram illustrating an example of a reflective object existing in a Z direction (depth direction).

FIG. 3 is a diagram illustrating an example of an amplitude of signal strength in the Z direction obtained in each pixel.

FIG. 4 is a block diagram illustrating correction processing according to a first embodiment.

FIG. 5 is a schematic diagram illustrating a schematic configuration example of a medical observation device according to a second embodiment.

FIG. 6 is a block diagram illustrating correction processing according to the second embodiment.

FIG. 7 is a schematic diagram illustrating a schematic configuration example of a medical observation device according to a third embodiment.

FIG. 8 is a schematic diagram illustrating a schematic configuration example of a medical observation device according to a fourth embodiment.

FIG. 9 is a schematic diagram illustrating a schematic configuration example of an observation device according to a fifth embodiment.

FIG. 10 is a schematic diagram illustrating a schematic configuration example of a medical observation device according to a sixth embodiment.

FIG. 11 is a diagram illustrating an example of a drive area of an image sensor according to the sixth embodiment.

FIG. 12 is a diagram illustrating an example of a volume acquisition time in each case where the number of pixels in the Y direction of the drive area is changed, according to the sixth embodiment.

FIG. 13 is a schematic diagram illustrating a modification of the medical observation device according to the sixth embodiment.

FIG. 14 is a schematic diagram illustrating a schematic configuration example of a medical observation device according to a seventh embodiment.

FIG. 15 is a diagram illustrating an example of an image rotator according to the seventh embodiment.

FIG. 16 is a diagram illustrating another example of the image rotator according to the seventh embodiment.

FIG. 17 is a schematic diagram illustrating a modification of the medical observation device according to the seventh embodiment.

FIG. 18 is a diagram illustrating an example of a drive area according to an eighth embodiment.

FIG. 19 is a schematic diagram illustrating a schematic configuration example of a medical observation device according to a ninth embodiment.

FIG. 20 is a schematic diagram illustrating a schematic configuration example of a medical observation device according to a tenth embodiment.

FIG. 21 is a partially enlarged view of a pixel array unit in a polarization image sensor according to the tenth embodiment.

FIG. 22 is a schematic diagram illustrating a schematic configuration example of a medical observation device according to an eleventh embodiment.

FIG. 23 is a block diagram illustrating an example of a hardware configuration according to an embodiment of the present disclosure.

### DESCRIPTION OF EMBODIMENTS

Embodiments of the present disclosure will be described below in detail with reference to the drawings. Note that, in each of the following embodiments, the same elements are denoted by the same reference symbols, and a repetitive description thereof will be omitted.

The present disclosure will be described in the following order of items.
1. First Embodiment
1.1 Schematic configuration example of medical observation device
1.2 Generation of tomographic image data
1.3 Correction processing
1.4 Summary
2. Second Embodiment
2.1 Schematic configuration example of medical observation device
2.2 Correction processing
3. Third Embodiment
4. Fourth Embodiment
5. Fifth Embodiment
6. Sixth Embodiment
6.1 Schematic configuration example of medical observation device
6.2 Summary
6.3 Modification
7. Seventh Embodiment
7.1 Modification
8. Eighth Embodiment
9. Ninth Embodiment
10. Tenth Embodiment
11. Eleventh Embodiment
12. Hardware configuration

### 1. First Embodiment

First, a medical observation device and an information processing device according to a first embodiment of the present disclosure will be described in detail with reference to the drawings. The present embodiment and embodiments to be described below will describe exemplary cases where the medical observation device and the information processing device according to each embodiment, as the medical observation device and the information processing device, are applied to a surgical microscope, a funduscope, or the like used in surgery or diagnosis of a human eye, such as glaucoma treatment. However, the present disclosure is not limited thereto, and is applicable to various observation devices in which light (reflected light, transmitted light, scattered light, and the like) from an examination object (subject) may have aberration.

### 1.1 Schematic Configuration Example of Medical Observation Device

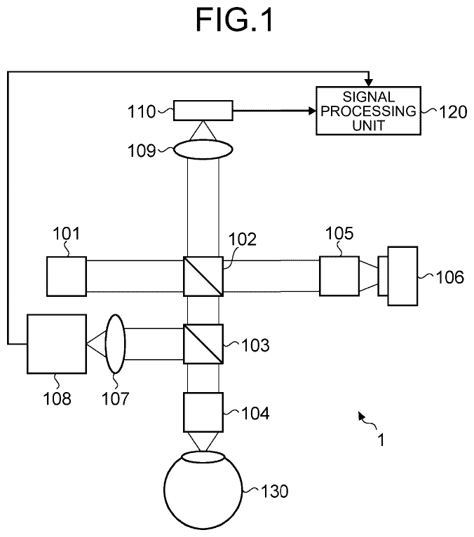

FIG. 1 is a schematic diagram illustrating a schematic configuration example of a medical observation device according to the present embodiment. As illustrated in FIG. 1, a medical observation device 1 according to the present embodiment includes an incoherent light source 101, beam splitters 102 and 103, objective lenses 104 and 105, a vibration mechanism 106, imaging lenses 107 and 109, a wavefront sensor 108, an image sensor 110, and a signal processing unit 120. The present embodiment assumes a case where a subject 130 as an examination object is a human eye, and a three-dimensional tomographic image of the human eye is acquired for the purpose of ophthalmic surgery or ophthalmic examination.

The incoherent light source 101 is a light source that emits at least spatially incoherent light (hereinafter, also referred to as spatially incoherent light), and may be various light sources that can emit spatially incoherent light, such as a halogen lamp, for example. By using the incoherent light source 101 as the light source of the medical observation device 1, the influence of the wavefront aberration by the subject 130 such as the human eye can be defined as reduction in the signal level (for example, equivalent to luminance or light intensity) instead of the reduction in the resolution. This makes it possible to suppress the reduction in the image quality of a three-dimensional tomographic image due to the reduction in the resolution. Although not illustrated, the incoherent light source 101 is supposed to include a collimator lens that collimates light emitted from the light source, for example.

The spatially incoherent light emitted from the incoherent light source 101 (hereinafter, also referred to as emitted light in order to be distinguished from other types of light) is incident on the beam splitter 102 and is split into two optical paths. The beam splitter 102 may include an optical element that transmits a part of light and reflects at least a part of the remaining light, such as a half mirror.

The emitted light transmitted through the beam splitter 102 is incident on a light incident surface of the vibration mechanism 106 via the objective lens 105, for example. The vibration mechanism 106 includes a piezoelectric element, for example. The light incident surface of the vibration mechanism 106 includes a reflection mirror that moves along the optical axis together with the vibration of the vibration mechanism 106. Accordingly, an optical path length of the light transmitted through the beam splitter 102 changes with the vibration of the vibration mechanism 106. At least a part of the light (hereinafter, also referred to as reference light) reflected by the reflection mirror of the vibration mechanism 106 is incident on the beam splitter 102 again and reflected, and forms an image on the image sensor 110 via an imaging lens 109 described below.

On the other hand, at least a part of the emitted light that is emitted from the incoherent light source 101 and reflected by the beam splitter 102 is incident on the subject 130 via the beam splitter 103 and the objective lens 104. The light reflected by the subject 130 (hereinafter, also referred to as observation light) is incident on the beam splitter 103 via the objective lens 104 and is split into two optical paths. Similarly to the beam splitter 102, the beam splitter 103 may include an optical element that transmits a part of the light and reflects at least a part of the remaining light, such as a half mirror.

The observation light reflected by the beam splitter 103 is incident on the wavefront sensor 108 via the imaging lens 107, for example. The wavefront sensor 108 detects the wavefront of the incident light, that is, the observation light reflected by the subject 130, and inputs the detection result (that is, a spatial distribution map of the wavefront aberration) to the signal processing unit 120.

On the other hand, at least a part of the observation light reflected by the subject 130 and transmitted through the beam splitter 103 is imaged on the image sensor 110 via the beam splitter 102 and the imaging lens 109.

Here, an optical axis of the observation light transmitted through the beam splitter 102 substantially matches an optical axis of the reference light reflected by the vibration mechanism 106 and reflected by the beam splitter 102. In addition, an optical path length from the beam splitter 102 to the vibration mechanism 106 when the light incident surface of the vibration mechanism 106 is at the reference position (for example, a position in a state where the vibration mechanism 106 is not vibrating) substantially matches an optical path length from the beam splitter 102 to the subject 130. Accordingly, the image formed on the light receiving surface of the image sensor 110 is an image having a pattern generated by interference of the observation light with the reference light. Therefore, by vibrating the light incident surface of the vibration mechanism 106 along the optical axis to vary the optical path length of the reference light, it is possible to acquire an image of the subject 130 along the optical axis (hereinafter, also referred to as a Z axis or a Z direction) (optical coherence tomography: OCT). The Z axis may be an axis parallel to the optical axis of the light incident on each of the incoherent light source 101, the vibration mechanism 106, the wavefront sensor 108, the image sensor 110, and the subject 130.

The image sensor 110 includes a pixel array unit, which includes a plurality of pixels that performs photoelectric conversion of incident light to generate luminance values (also referred to as pixel values), with the plurality of pixels arranged in a two-dimensional matrix. The image sensor 110 outputs two-dimensional image data (hereinafter, also simply referred to as image data) of an image generated by the interference of the beams of incident light, namely, the observation light and the reference light. That is, the medical observation device 1 according to the present embodiment may be configured as a full-field OCT (FFOCT) capable of acquiring a three-dimensional tomographic image of the subject 130 without requiring scanning in the horizontal direction (XY plane direction). Therefore, the image sensor 110 outputs image data at a predetermined frame rate during vibration of the vibration mechanism 106 (that is, during movement of the reflection mirror along the optical axis (Z axis)), enabling acquisition of a three-dimensional tomographic image of the subject 130.

The signal processing unit 120 generates a three-dimensional tomographic image of the subject 130 using the image data input from the image sensor 110 at a predetermined frame rate. Specifically, for example, the amplitude of the vibration of the signal level in each pixel in several frames in the vicinity in the Z direction is calculated as a signal level indicating the reflection intensity (corresponding to the luminance or the light intensity of the observation light) on a tomographic region at the Z-axis position. This generates tomographic image data of the tomographic region at the Z-axis position. By stacking the generated tomographic image data in the Z direction, a three-dimensional tomographic image is generated. At that time, the signal processing unit 120 corrects the signal level of the tomographic image data used for generating the three-dimensional tomographic image based on a spatial distribution map of the wavefront aberration (hereinafter, also referred to as a spatial aberration map). This makes it possible to correct a signal level reduced due to the influence of the wavefront aberration generated in the subject 130 such as a human eye, leading to achievement of suppression of image quality degradation of the three-dimensional tomographic image due to the reduction of the signal level.

The detection of the spatial aberration map by the wavefront sensor 108 may be executed every time the signal processing unit 120 generates the three-dimensional tomographic image, or may be executed in a case where the positional relationship between the subject 130 and the objective lens 104 is changed. In the latter case, the spatial aberration map acquired in advance can be used for generation of the subsequent three-dimensional tomographic image in a state where the positional relationship between the subject 130 and the objective lens 104 has not been changed. This makes it possible to reduce the processing volume and the processing time in subsequent three-dimensional tomographic image generation processing. Incidentally, the occurrence of a change in the positional relationship between the subject 130 and the objective lens 104 may be manually input by the user, may be detected using a sensor provided in the subject 130 and/or the objective lens 104, or may be determined based on execution of initialization processing manually or automatically after adjustment of the positional relationship between the subject 130 and the objective lens 104, for example.

### 1.2 Generation of Tomographic Image Data

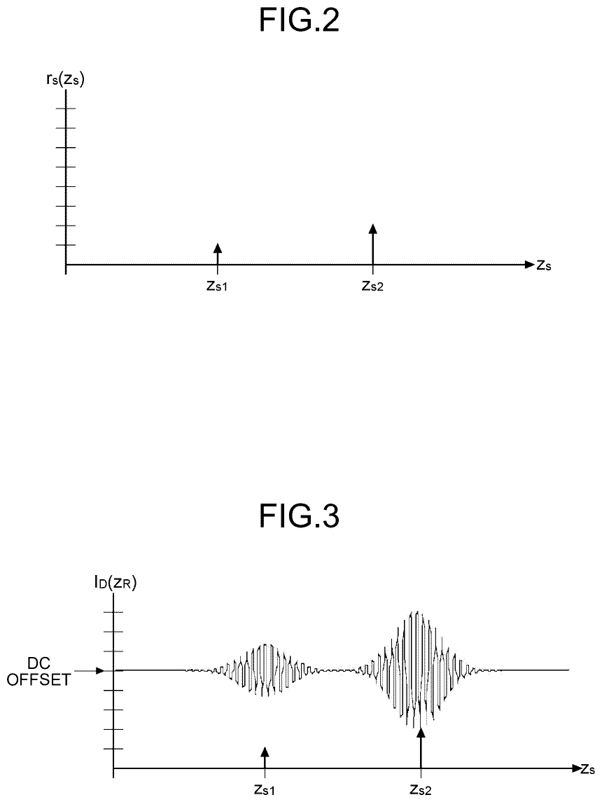

As described above, in the present embodiment, the tomographic image data is generated based on the image data obtained by scanning an interference pattern created by observation light and reference light in the Z direction (also referred to as the depth direction in the present description). Here, as illustrated in FIG. 2, in a case where there are reflective objects at each of two points in the Z direction, the strength of a signal obtained as a result of scanning in the Z direction for a certain pixel on the image sensor 110 indicates vibration in the Z direction due to interference of observation light and reference light as illustrated in FIG. 3. The amplitude of this vibration represents the reflectance of the object. That is, the magnitude of the amplitude represents the luminance of the observation image reflected by the reflective object.

Accordingly, the signal processing unit 120 calculates an amplitude value at each position in the Z direction as a signal level of each pixel at the position in the Z direction, thereby generating tomographic image data at the Z-axis position.

### 1.3 Correction Processing

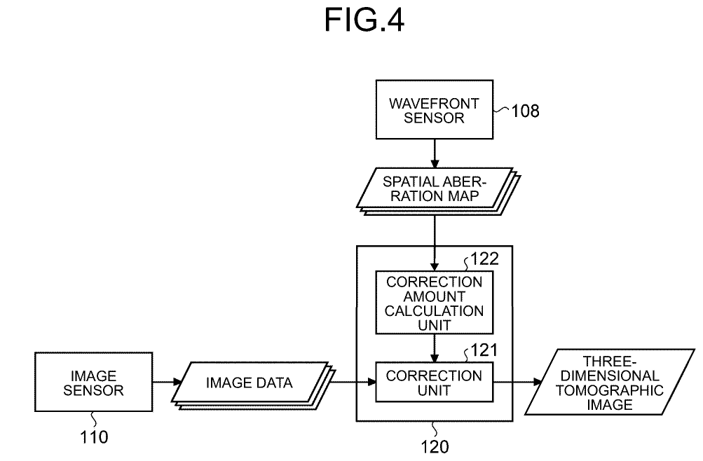

Next, correction processing of the signal level based on the spatial aberration map performed by the signal processing unit 120 will be described. FIG. 4 is a block diagram illustrating correction processing according to the present embodiment. Here is an exemplary case where the detection of the spatial aberration map is executed in the wavefront sensor 108 every time the three-dimensional tomographic image is generated in the signal processing unit 120.

As illustrated in FIG. 4, the signal processing unit 120 includes a correction unit 121 and a correction amount calculation unit 122.

The signal processing unit 120 receives an input of two-dimensional image data from the image sensor 110 to at a predetermined frame rate and an input of a spatial aberration map from the wavefront sensor 108. The signal processing unit 120 generates tomographic image data in which a signal level is used to represent a pixel value, based on image data input at a predetermined frame rate.

The generated tomographic image data is input to the correction unit 121. On the other hand, the spatial aberration map input to the signal processing unit 120 is input to the correction amount calculation unit 122. Incidentally, the spatial aberration map may be input from the wavefront sensor 108 in parallel with the input of the image data from the image sensor 110, or may be input from the wavefront sensor 108 before or after the input of the image data from the image sensor 110.

The correction amount calculation unit 122 calculates the correction amount of the signal level in each region (for example, at each pixel) in the tomographic image data based on the spatial aberration map input from the wavefront sensor 108. For example, the correction amount calculation unit 122 calculates a correction amount such that the correction amount (for example, the amplification amount) of the signal level is increased in a region with large wavefront aberration and the correction amount (for example, the amplification amount) of the signal level is decreased in a region with small wavefront aberration. However, the calculation of the correction amount is not limited thereto. The correction amount that increases the signal level may be calculated in the region with large wavefront aberration, and the correction amount that reduces the signal level may be calculated in the region with small wavefront aberration. Still alternatively, the correction amount may be calculated such that the correction amount (for example, decreasing amount) of the signal level is small in the region with large wavefront aberration and the correction amount (for example, decreasing amount) of the signal level is large in the region with small wavefront aberration.

The correction amount calculated for each region is input to the correction unit 121. The correction unit 121 corrects the signal level of each region in the tomographic image data based on the correction amount input from the correction amount calculation unit 122. The correction unit 121 then generates a three-dimensional tomographic image using the corrected tomographic image data, and outputs the generated three-dimensional tomographic image to the outside.

In this manner, the wavefront aberration generated by the subject 130 is actually measured, and the signal level of the tomographic image data is corrected based on the spatial aberration map as a result of the measurement, thereby improving the image quality of the tomographic image data used for generating the three-dimensional tomographic image. This makes it possible to generate a three-dimensional tomographic image with higher image quality.

### 1.4 Summary

As described above, the medical observation device 1 according to the present embodiment includes: the incoherent light source 101 that emits at least spatially incoherent light; the image sensor 110 that acquires two-dimensional image data of the interference pattern created by the observation light emitted from the incoherent light source 101 and reflected by the subject 130 and the predetermined reference light; the wavefront sensor 108 that measures the spatial aberration map indicating how the influence of the wavefront aberration of the subject 130 is spatially distributed by detecting the observation light; and the signal processing unit 120 that corrects the signal level of the tomographic image data obtained from the image data of the interference pattern based on the spatial aberration map obtained by measurement.

In this manner, by using the incoherent light source 101 as a light source at the time of photographing the interference pattern, the influence of the wavefront aberration of the subject 130 such as the human eye can be defined as reduction in the signal level, rather than reduction in the resolution. Furthermore, by using the wavefront sensor 108, it is possible to measure how the influence of the wavefront aberration of the subject 130 is spatially distributed. By combining individual data, it is possible to grasp the degree of spatially occurring signal level reduction, making it possible to correct the signal level reduction due to the wavefront aberration by performing signal processing on the tomographic image data obtained from the image data. As a result, it is possible to generate a three-dimensional tomographic image with high resolution at low cost without using an expensive optical device such as a spatial optical phase modulator. With the capability of generating a high-quality three-dimensional tomographic image, it is possible to improve recognition accuracy by visual observation, artificial intelligence (AI), or the like, leading to acquisition of recognition results with higher precision with a short time.

In addition, by applying the medical observation device 1 having the above-described configuration to a surgical microscope, a funduscope, or the like, it is possible to increase the commonality of many optical components ranging from an eyepiece lens to an image sensor. This makes it possible to simplify and downsize the equipment configuration of the entire medical device including the surgical microscope or the funduscope.

### 2. Second Embodiment

Next, a medical observation device and an information processing device according to a second embodiment of the present disclosure will be described in detail with reference to the drawings. In the following description, the configuration, operation, and effects similar to those of the above-described embodiments will be cited, thereby omitting redundant description.

### 2.1 Schematic Configuration Example of Medical Observation Device

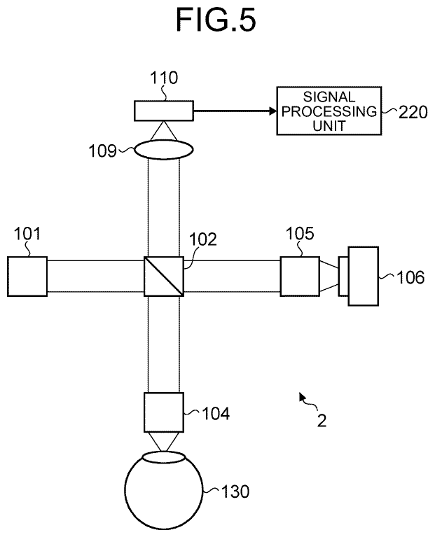

FIG. 5 is a schematic diagram illustrating a schematic configuration example of a medical observation device according to the present embodiment. As illustrated in FIG. 5, a medical observation device 2 according to the present embodiment has a configuration similar to the medical observation device 1 of the first embodiment described with FIG. 1, but in this configuration, the beam splitter 103, the imaging lens 107, and the wavefront sensor 108 are omitted, and the signal processing unit 120 is replaced with a signal processing unit 220.

Regarding the signal processing unit 220, in a case where the incoherent light source 101 is used as the light source, the influence of the aberration of the subject 130 such as an eye appears in tomographic image data as signal level reduction. Accordingly, the present embodiment specifies how much the signal level of which region has decreased, and corrects the signal level of each region (for example, each pixel) in the tomographic image data based on the degree of decrease in the signal level for each specified region.

As a method of calculating the correction amount for each region, for example, various methods may be adopted, such as preliminarily imaging a region with less texture in the subject 130 such as internal portions of the eyeball, and calculating or estimating the correction amount of each region based on the signal level of each region in the tomographic image data, obtained from the image data captured by the imaging.

### 2.2 Correction Processing

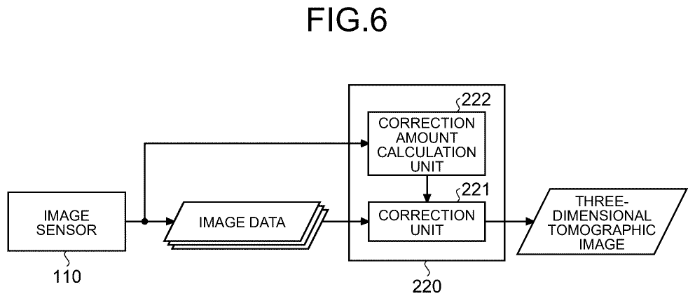

Next, correction processing by the signal processing unit 220 will be described. FIG. 6 is a block diagram illustrating correction processing according to the present embodiment. Here is an exemplary case where of preliminarily imaging a region with less texture in the subject 130 such as internal portions of the eyeball, and calculating the correction amount of each region based on the signal level of each region in the tomographic image data, obtained from the image data captured by the imaging.

As illustrated in FIG. 6, the signal processing unit 220 includes a correction unit 221 and a correction amount calculation unit 222. Here, the correction unit 221 may be similar to the correction unit 121 according to the first embodiment.

The signal processing unit 220 receives an input of image data of a region with less texture in the subject 130 preliminarily captured and an input of image data output from the image sensor 110 at a predetermined frame rate. The signal processing unit 220 generates tomographic image data (also referred to as first tomographic image data) in advance from the preliminarily captured image data of a region with less texture. Furthermore, the signal processing unit 220 generates tomographic image data (also referred to as second tomographic image data) from the image data output at a predetermined frame rate.

The first tomographic image data obtained from the preliminarily captured image data is input to the correction amount calculation unit 222, and the second tomographic image data obtained from the image data output at a predetermined frame rate is input to the correction unit 221. Note that which region in the subject 130 has less texture may be manually searched by the operator, or may be automatically specified by recognition processing or the like on one or more pieces of image data or the first tomographic image data covering the entire internal portions of the eyeball.

Based on the preliminarily obtained first tomographic image data, the correction amount calculation unit 222 calculates the correction amount of the signal level in each region (for example, each pixel) in the second tomographic image data obtained from the image data output at the predetermined frame rate. For example, with the maximum value of the signal level in the entire first tomographic image data as a reference, the correction amount calculation unit 222 may calculate the correction amount such that the more the signal level is lower, in a region, than the maximum value of the entire data, the larger the correction amount (for example, the amplification amount) of the signal level for that region. Calculation of correction amount is not limited thereto, and various modifications may be made such as calculating the correction amount based on the average value or the minimum value of the signal levels in the entire first tomographic image data.

The correction amount calculated for each region is input to the correction unit 221. Similarly to the correction unit 121 according to the first embodiment, the correction unit 221 corrects the signal level of each region in the second tomographic image data, obtained from the image data input from the image sensor 110 at a predetermined frame rate, based on the correction amount input from the correction amount calculation unit 222. The correction unit 221 then generates a three-dimensional tomographic image using the corrected second tomographic image data, and outputs the generated three-dimensional tomographic image to the outside.

In this manner, by actually measuring the decrease in the signal level caused by the subject 130 and correcting the signal level of the second tomographic image data based on a measurement result, it is possible to improve the image quality of the tomographic image data used for generating the three-dimensional tomographic image. This makes it possible to generate a three-dimensional tomographic image with higher image quality.

Since other configurations, operations, and effects may be similar to those in the above-described embodiment, detailed description will be omitted here.

### 3. Third Embodiment

Next, a medical observation device and an information processing device according to a third embodiment of the present disclosure will be described in detail with reference to the drawings. In the following description, the configuration, operation, and effects similar to those of the above-described embodiments will be cited, thereby omitting redundant description.

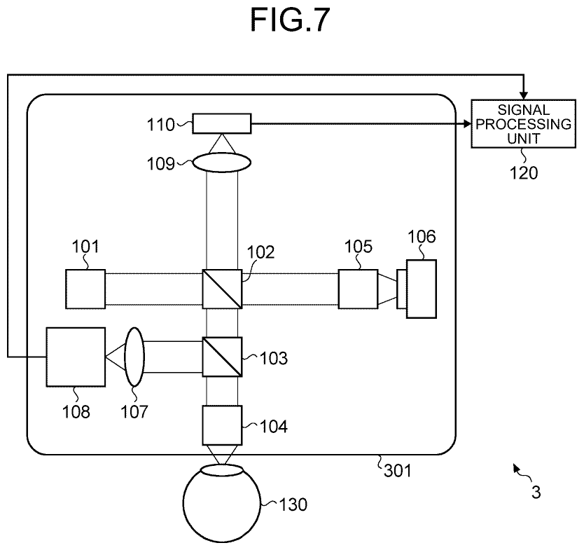

FIG. 7 is a schematic diagram illustrating a schematic configuration example of the medical observation device according to the present embodiment. As illustrated in FIG. 7, a medical observation device 3 according to the present embodiment has a configuration similar to the medical observation device 1 described with reference to FIG. 1 in the first embodiment, but in this configuration, the incoherent light source 101, the beam splitters 102 and 103, the objective lenses 104 and 105, the vibration mechanism 106, the imaging lenses 107 and 109, the wavefront sensor 108, and the image sensor 110 are fixed to a stage 301, which is movable in at least one direction out of the X direction, the Y direction, and the Z direction. Incidentally, as described above, the Z direction may be a direction parallel to the optical axis (Z axis), and both the X direction and the Y direction may each be a direction perpendicular to the Z direction. For example, the X direction may be parallel to the row direction of the pixel array in the pixel array unit of the image sensor 110, while the Y direction may be parallel to the column direction of the pixel array in the pixel array unit.

In this manner, with the configuration having the measurement system including the incoherent light source 101, the beam splitters 102 and 103, the objective lenses 104 and 105, the vibration mechanism 106, the imaging lenses 107 and 109, the wavefront sensor 108, and the image sensor 110 mounted on the movable stage 301, the relative position between the subject 130 and the measurement system can be changed. This makes it possible to change the measurement region in the subject 130 or to scan the measurement system to acquire image data of the subject 130 in a region (including the entire region) wider than one shot. The wavefront sensor 108 may detect the spatial aberration map in accordance with the movement of the stage 301. With this configuration, even when the subject 130 in a region (including the entire region) wider than one shot is measured, it is possible to acquire the spatial aberration map of the entire measurement region.

Since other configurations, operations, and effects may be similar to those in the above-described embodiment, detailed description will be omitted here.

### 4. Fourth Embodiment

Next, a medical observation device and an information processing device according to a fourth embodiment of the present disclosure will be described in detail with reference to the drawings. In the following description, the configuration, operation, and effects similar to those of the above-described embodiments will be cited, thereby omitting redundant description.

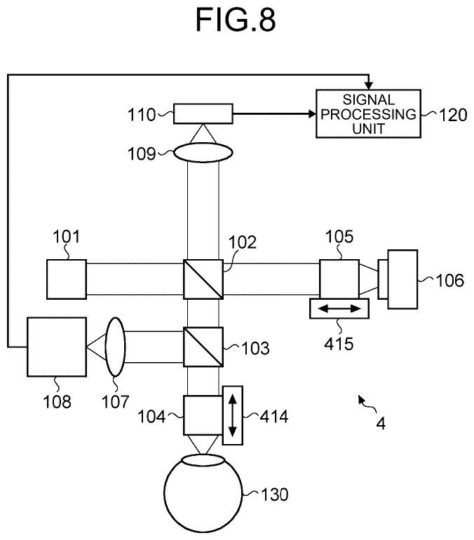

FIG. 8 is a schematic diagram illustrating a schematic configuration example of the medical observation device according to the present embodiment. As illustrated in FIG. 8, a medical observation device 4 according to the present embodiment has a configuration similar to the medical observation device 1 described with reference to FIG. 1 in the first embodiment, but this configuration has an additional mechanisms, namely, a moving mechanism 414 that moves the objective lens 104 facing the subject 130 along the optical axis and a moving mechanism 415 that moves the objective lens 105 facing the vibration mechanism 106 along the optical axis.

In this manner, by moving each of the objective lenses 104 and 105 along each optical axis, the focal position of the emitted light in the subject 130 can be moved in the Z direction, making it possible to change the measurement region in the subject 130 in the Z direction. This makes it possible to measure the subject 130 in a wider region (including the entire region) in the depth direction (Z direction). Incidentally, the movement of the objective lens 104 in the Z direction by the moving mechanism 414 and the movement of the objective lens 105 in the Z direction by the moving mechanism 415 may be synchronized to each other.

Since other configurations, operations, and effects may be similar to those in the above-described embodiment, detailed description will be omitted here.

### 5. Fifth Embodiment

Next, an observation device and an information processing device according to a fifth embodiment of the present disclosure will be described in detail with reference to the drawings. In the following description, the configuration, operation, and effects similar to those of the above-described embodiments will be cited, thereby omitting redundant description.

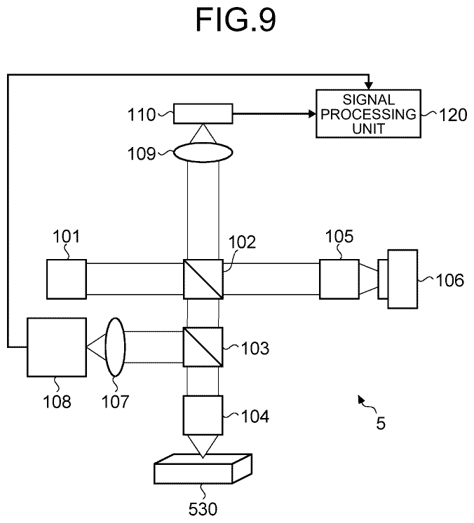

FIG. 9 is a schematic diagram illustrating a schematic configuration example of the observation device according to the present embodiment. In the above-described embodiment, the human eye is exemplified as the subject 130. However, the present disclosure is not limited thereto, and for example, as in a observation device 5 illustrated in FIG. 9, various objects having a possibility of having wavefront aberration, such as glass products including lenses and holograms, can be used as the subject 530.

Since other configurations, operations, and effects may be similar to those in the above-described embodiment, detailed description will be omitted here.

### 6. Sixth Embodiment

Next, a medical observation device and an information processing device according to a sixth embodiment of the present disclosure will be described in detail with reference to the drawings. In the following description, the configuration, operation, and effects similar to those of the above-described embodiments will be cited, thereby omitting redundant description.

As in the above-described embodiment, in a time domain FFOCT in which a three-dimensional tomographic image of the subject 130 or 530 (hereinafter, the subject 130 will be used as an example) is acquired by moving the reflection mirror in the Z direction by the vibration mechanism 106, an OCT image of the xy plane (hereinafter, the image is also referred to as an en-face image) can be acquired at high speed. However, an OCT image of the XZ plane (B-scan image) needs to be created by preliminarily acquiring three-dimensional volume data, which is a stack of en-face images in the Z direction, and clipping three-dimensional volume data on a necessary plane from the stack, making it difficult to acquire the B-scan image at high speed.

To handle this, in the present embodiment, a medical observation device and an information processing device capable of reducing the time required to acquire the B-scan image will be described with an example. The present embodiment, the following embodiment, and the modifications thereof will describe an exemplary case where the medical observation device is constructed as a time domain type FFOCT similarly to the above-described embodiment. However, the device type is not limited thereto, and the medical observation device can be configured as a wavelength sweep type FFOCT.

### 6.1 Schematic Configuration Example of Medical Observation Device

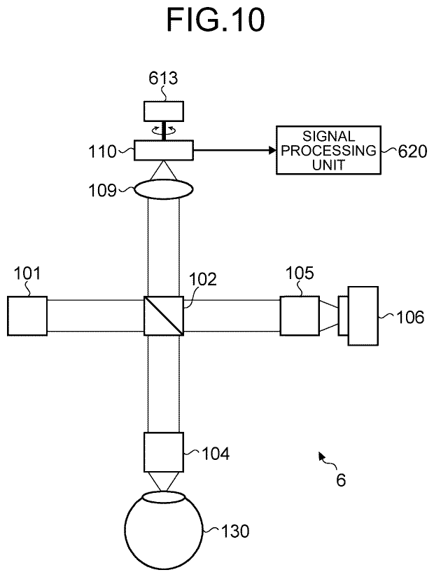

FIG. 10 is a schematic diagram illustrating a schematic configuration example of the medical observation device according to the present embodiment. As illustrated in FIG. 10, a medical observation device 6 according to the present embodiment has a configuration similar to the medical observation device 1 described with reference to FIG. 1 in the first embodiment, but this configuration additionally includes a rotation mechanism 613 for allowing pivot movement of the image sensor 110 about the optical axis (Z axis) as a rotation axis, and includes a signal processing unit 620 in place of the signal processing unit 120. The rotation mechanism 613 may be an example of an adjustment mechanism which adjusts the rotation angle of the image sensor with respect to the image of the observation light, with the optical axis of the observation light defined as a rotation axis.

FIG. 10 illustrates a case where the optical system (beam splitter 103, imaging lens 107, and wavefront sensor 108) for detecting the wavefront of the observation light is omitted. However, the configuration is not limited thereto, and the signal processing unit 620 may correct the signal level of a tomographic image data based on a spatial aberration map input from the wavefront sensor 108, similarly to the signal processing unit 120 according to the above-described embodiment.

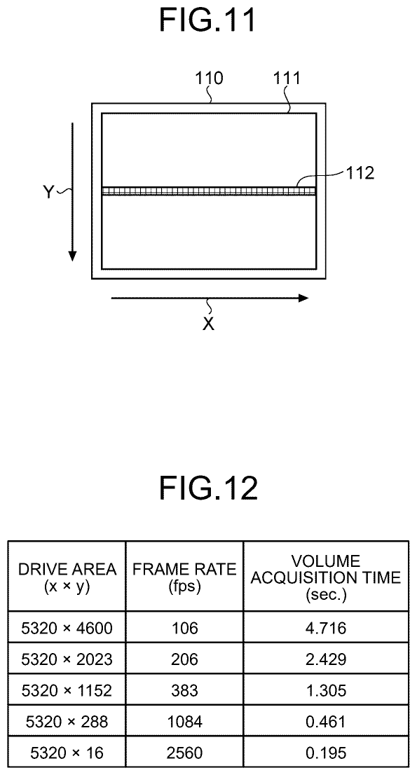

FIG. 11 is a diagram illustrating an example of a drive area of an image sensor according to the present embodiment. As illustrated in FIG. 11, in the present embodiment, in order to directly acquire a necessary B-scan image of the XZ plane, an area (also referred to as a drive area) 112 to be read by the image sensor 110 is narrowed down. For example, in a case where the image sensor 110 includes a pixel array unit 111 in which a plurality of pixels is two-dimensionally arranged in a matrix, and uses a rolling shutter method of reading image data with pixels arranged in a line in the X direction as a unit (hereinafter, also referred to as a row unit or a line), the drive area 112 may be a rectangular region formed of one or several lines and long in the X direction, for example. However, the present invention is not limited thereto, and the drive area 112 may be a rectangular region long in the Y direction, or may be a rectangular region long in a direction inclined with respect to the X and Y directions. Furthermore, the driving method of the image sensor 110 is not limited to the rolling shutter method, and may be a global shutter method capable of simultaneously reading all pixels. For the sake of simplicity, the following will describe an exemplary case where the drive area 112 is a rectangular region long in the X direction.

FIG. 12 is a diagram illustrating an example of a volume acquisition time in each case where the number of pixels in the Y direction of the drive area is changed, according to the present embodiment. As illustrated in FIG. 12, the smaller the number of pixels in the Y direction of the drive area 112 in the image sensor 110, the shorter the reading time at the time of reading one en-face image can be, making it possible to increase the frame rate. As a result, it is possible to shorten the acquisition time (volume acquisition time) of three-dimensional volume data, which is a stack of the en-face images in the Z direction.

For example, in a case where the number of pixels in the Y direction of the drive area 112 is 4600 pixels, the volume acquisition time is 4.716 seconds. However, by setting the number of pixels in the Y direction of the drive area 112 to 16 pixels, the volume acquisition time can be shortened to 0.195 seconds.

The image data (en-face image) long in the X direction output from the image sensor 110 is input to the signal processing unit 620. Here, the three-dimensional volume data, which is a Z-direction stack of the en-face images long in the X direction, substantially corresponds to a B-scan image obtained by slicing the three-dimensional volume data of the subject 130 at the XZ plane. Accordingly, in the present embodiment, the B-scan image of the subject 130 is generated by performing Z-direction stacking of the tomographic image data obtained from the image data output from the image sensor 110.

In this manner, by forming the drive area 112 in the image sensor 110 into a rectangular region, it is possible to directly acquire the B-scan image of the subject 130. In addition, the volume acquisition time can be shortened with the smaller number of pixels in the lateral direction of the drive area 112, leading to acquisition of the B-scan image at high speed.

Furthermore, in the present embodiment, by using the rotation mechanism 613, the image sensor 110 can be rotated with the optical axis as a rotation axis, for example. Accordingly, the present embodiment makes it possible to acquire the B-scan image on an optionally determined plane passing through the optical axis by rotating the image sensor 110 using the rotation mechanism 613. Note that the rotation of the image sensor 110 by the rotation mechanism 613 may be performed under the control of a control unit (not illustrated) or the like, or this control may be performed based on an operation input from a user such as an operator.

Furthermore, for example, as exemplified in the third embodiment, with a configuration in which a measurement system including the image sensor 110 is mounted on the stage 301 to make the system movable, it is also possible to acquire a B-scan image of an optionally determined XZ plane of the subject 130.

### 6.2 Summary

As described above, according to the present embodiment, since the drive area 112 in the image sensor 110 is limited to a rectangular region long in one direction, it is possible to directly acquire the B-scan image at high speed. This makes it possible to shorten the surgery time and the examination time for the subject 130, leading to reduction of the burden on the subject 130.

Furthermore, by making the image sensor 110 rotatable, it is possible to acquire a B-scan image of an optionally determined XZ plane passing through the optical axis. Furthermore, with the movable measurement system including the image sensor 110, it is also possible to acquire a B-scan image of an optionally determined XZ plane of the subject 130.

Since other configurations, operations, and effects may be similar to those in the above-described embodiment, detailed description will be omitted here.

### 6.3 Modification

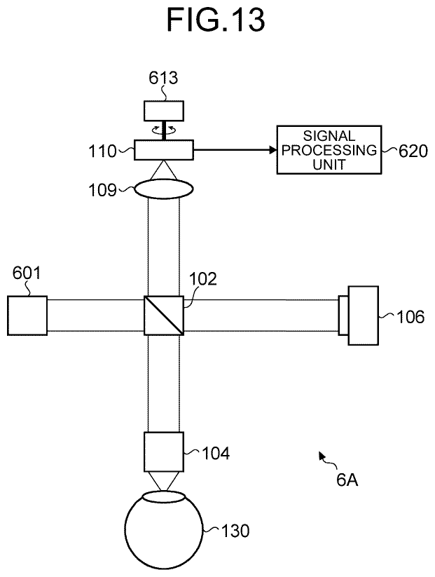

FIG. 13 is a schematic diagram illustrating a modification of the medical observation device according to the sixth embodiment. The above sixth embodiment has described an exemplary case where the incoherent light source 101 is used as the light source. However, the light source according to the present embodiment is not limited thereto, and may be a coherent light source 601 that emits at least spatially coherent light (hereinafter, also referred to as spatial coherent light) as in a medical observation device 6A illustrated in FIG. 13. The coherent light source 601 may be implemented by using a Superluminescent Diode (SLD) or the like.

In this manner, by using the coherent light source 601 as the light source, as illustrated in FIG. 13, it is possible to omit the objective lens 105 arranged on the light incident surface of the vibration mechanism 106, leading to simplification of the optical system of the medical observation device 6A.

Since other configurations, operations, and effects may be similar to those in the above-described embodiment, detailed description will be omitted here.

### 7. Seventh Embodiment

Next, a medical observation device and an information processing device according to a seventh embodiment of the present disclosure will be described in detail with reference to the drawings. In the following description, the configuration, operation, and effects similar to those of the above-described embodiments will be cited, thereby omitting redundant description.

In the above-described sixth embodiment and the modifications thereof, the XZ plane for acquiring the B-scan image is changed by rotating the image sensor 110 using the rotation mechanism 613. In contrast, the present embodiment will describe an exemplary case where the XZ plane used for acquisition of the B-scan image is changed by rotating an image formed on the image sensor 110.

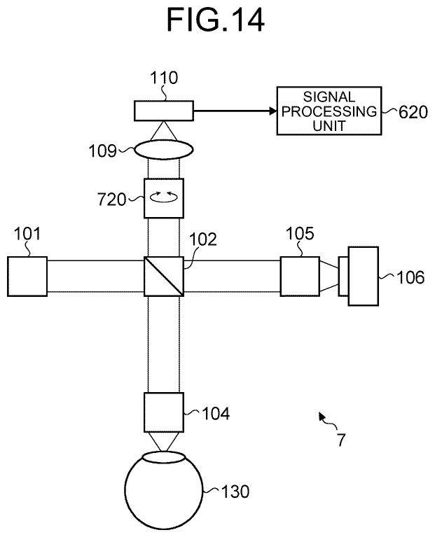

FIG. 14 is a schematic diagram illustrating a schematic configuration example of the medical observation device according to the present embodiment. As illustrated in FIG. 14, a medical observation device 7 according to the present embodiment has a configuration similar to the medical observation device 6 described with reference to FIG. 10 in the sixth embodiment, but this configuration omits the rotation mechanism 613 and includes, as a replacement, an image rotator 720 that rotates an image about an optical axis and is disposed on an optical path from the beam splitter 102 to the imaging lens 109. The image rotator 720 may be an example of an adjustment mechanism that adjusts the rotation angle of the image sensor with respect to the image of the observation light, with the optical axis of the observation light defined as a rotation axis. Also in the present embodiment, similarly to the sixth embodiment or the modification thereof, the rectangular drive area 112 long in one direction is set in the image sensor 110.

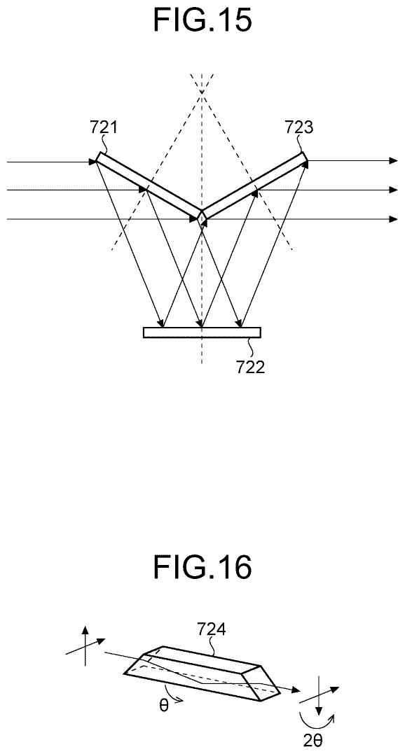

FIGS. 15 and 16 are diagrams illustrating an example of an image rotator according to the present embodiment. The image rotator 720 according to the present embodiment can be implemented by adopting various types of devices, such as one including a plurality of (for example, three) mirrors 721 to 723 as illustrated in FIG. 15, or one using a dove prism 724 as illustrated in FIG. 16.

In this manner, by rotating an image incident on the image sensor 110, instead of rotating the image sensor 110, it is possible to omit the rotation mechanism 613, leading to the simplified configuration of the medical observation device 7.

Since other configurations, operations, and effects may be similar to those in the above-described embodiment, detailed description will be omitted here.

### 7.1 Modification

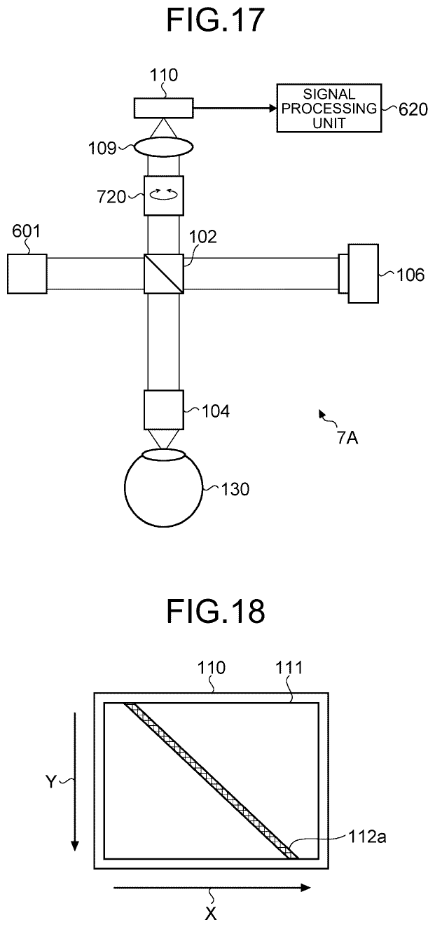

FIG. 17 is a schematic diagram illustrating a modification of the medical observation device according to the seventh embodiment. The above seventh embodiment has described an exemplary case where the incoherent light source 101 is used as the light source. However, the light source according to the present embodiment is not limited thereto, and a coherent light source 601 such as an SLD may be used similarly to the modification of the sixth embodiment. By using the coherent light source 601 as the light source, it is possible to omit the objective lens 105 arranged on the light incident surface of the vibration mechanism 106, leading to simplification of the optical system of a medical observation device 7A.

Since other configurations, operations, and effects may be similar to those in the above-described embodiment, detailed description will be omitted here.

### 8. Eighth Embodiment

Next, a medical observation device and an information processing device according to an eighth embodiment of the present disclosure will be described in detail with reference to the drawings. In the following description, the configuration, operation, and effects similar to those of the above-described embodiments will be cited, thereby omitting redundant description.

FIG. 18 is a diagram illustrating an example of a drive area according to the present embodiment. The above-described sixth and seventh embodiments and the modifications thereof are an exemplary case where the drive area 112 is fixed in the image sensor 110. However, for example, in a case where an image sensor using a global shutter method is adopted as the image sensor 110, as illustrated in FIG. 18, it is possible to freely change the drive area 112a while suppressing redundancy of the read time. This makes it possible to acquire a B-scan image of an optionally determined XZ plane without requiring the rotation mechanism 613, the image rotator 720, or the stage 301.

Since other configurations, operations, and effects may be similar to those in the above-described embodiment, detailed description will be omitted here.

### 9. Ninth Embodiment

Next, a medical observation device and an information processing device according to a ninth embodiment of the present disclosure will be described in detail with reference to the drawings. In the following description, the configuration, operation, and effects similar to those of the above-described embodiments will be cited, thereby omitting redundant description. The following will describe an exemplary case where the medical observation device 6 described with reference to FIG. 10 in the sixth embodiment is used as a base. However, the configuration is not limited thereto, and it is also possible to use a medical observation device according to another embodiment or a modification thereof as a base.

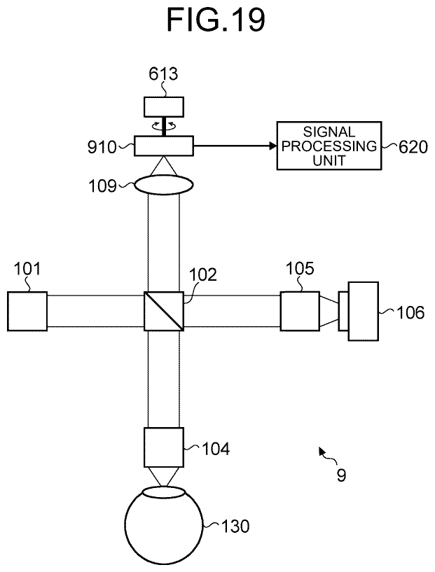

FIG. 19 is a schematic diagram illustrating a schematic configuration example of the medical observation device according to the present embodiment. The above embodiment has described an exemplary case of using the image sensor 110 that detects light in the visible light spectrum to generate image data, as the image sensor that images an interference pattern created by the observation light and the reference light. However, the configuration is not limited thereto. For example, as in a medical observation device 9 illustrated in FIG. 19, it is also possible to use an image sensor 910 that generates image data by detecting light of a spectrum other than the visible light spectrum, such as short wavelength infra-red (SWIR) instead of or in addition to light in the visible light spectrum. Assuming that the light receiving sensitivity of the image sensor 110 that detects visible light is in a wavelength band around 400 nanometers (nm) to 900 nm, the light receiving sensitivity of the image sensor 910 capable of detecting SWIR light may be in a wavelength band around 400 nm to 1700 nm.

Wavelength bands of light used in typical OCT include an 850 nm band, a 1 micrometer (μm) band, and a 1.3 μm band. Here, the light in the 850 nm band is used in observation of the anterior eye portion and the fundus portion, for example, and using the use of light having a longer wavelength (for example, 1 μm band light) can reduce scattering, leading to improved tissue depth achievement level in the fundus portion. In addition, light in a 1.3 μm band is used to observe the anterior eye portion.

Accordingly, as in the present embodiment, by using the image sensor 910 capable of observing light having a longer wavelength, it is possible to provide the medical observation device 9 capable of performing observation with higher accuracy. For example, by using the image sensor 910 capable of observing light in the 1.0 μm band, it is possible to improve the tissue depth achievement level in the fundus oculi. In addition, by using the image sensor 910 capable of observing light in the 1.3 μm band, it is possible to improve the clarity of the OCT image of the anterior eye portion. Furthermore, by increasing the wavelength width of the light receiving sensitivity of the image sensor 910, the resolution in the axial direction can be improved.

In this manner, with the present embodiment, it is possible to select a wavelength or a wavelength width according to the observation target (for example, human cell tissue), and making it possible to acquire an image with higher clarity. This makes it possible to obtain an appropriate diagnosis result.

Since other configurations, operations, and effects may be similar to those in the above-described embodiment, detailed description will be omitted here.

### 10. Tenth Embodiment

Next, a medical observation device and an information processing device according to a tenth embodiment of the present disclosure will be described in detail with reference to the drawings. In the following description, the configuration, operation, and effects similar to those of the above-described embodiments will be cited, thereby omitting redundant description. The following will describe an exemplary case where the medical observation device 6 described with reference to FIG. 10 in the sixth embodiment is used as a base. However, the configuration is not limited thereto, and it is also possible to use a medical observation device according to another embodiment or a modification thereof as a base.

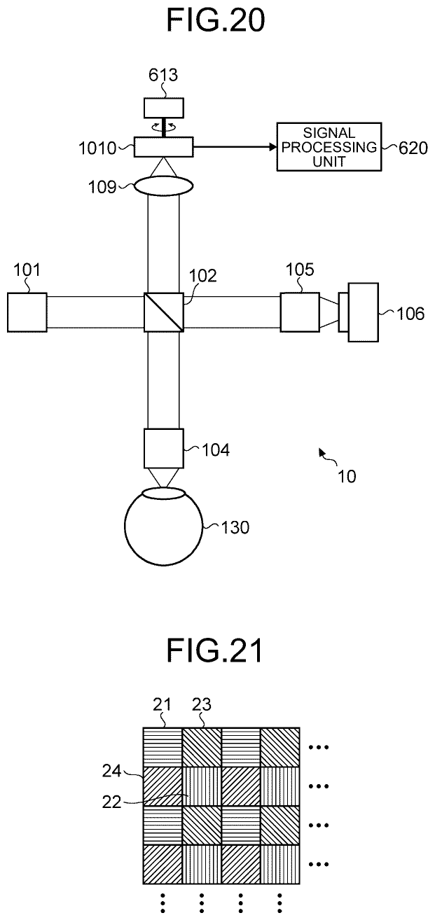

FIG. 20 is a schematic diagram illustrating a schematic configuration example of the medical observation device according to the present embodiment. FIG. 21 is a partially enlarged view of a pixel array unit in a polarization image sensor according to the present embodiment.

As illustrated in FIG. 20, for example, a medical observation device 10 according to the present embodiment has a configuration similar to the medical observation device 6 described with reference to FIG. 10 in the sixth embodiment, but this configuration includes a polarization image sensor 1010 in place of the image sensor 110.

As illustrated in FIG. 21, the polarization image sensor 1010 has a configuration in which pixels 21 to 24 are arranged in 2×2 array patterns, and this pattern is repeated in row/column directions, that is, in a matrix, for example. Specifically, the pixel 21 has light receiving sensitivity to light polarized in a lateral direction (for example, in the X direction) in the drawing, the pixel 22 has light receiving sensitivity to light polarized in a longitudinal direction (for example, in the Y direction), the pixel 23 having light receiving sensitivity to light polarized in a direction diagonally upward to the left, and the pixel 24 has light receiving sensitivity to light polarized in a direction diagonally upward to the right.

A typical OCT uses an image sensor having no polarization dependency. Therefore, in order to acquire an interference pattern for each polarization, it has been necessary to split p-polarized waves and s-polarized waves by a polarization beam splitter so as to acquire the interference pattern with two image sensors. In comparison, by using the polarization image sensor 1010 having an array of pixels having light receiving sensitivity to light polarized in different directions as in the present embodiment, it is possible to acquire an interference pattern for each polarization direction in one shot without requiring a polarization beam splitter or two image sensors. Incidentally, FIG. 21 illustrates the polarization image sensor 1010 capable of acquiring interference patterns in each of the four polarization directions of the horizontal direction, the vertical direction, the direction diagonally upward to the left, and the direction diagonally upward to the right in one shot. However, the present embodiment is not limited to this, and the directions may be altered in various manners, such as using two polarization directions of the horizontal direction and the vertical direction, for example.

In this manner, by adopting a configuration in which interference patterns in a plurality of polarization directions can be acquired in one shot, it is possible to acquire an image reflecting birefringence while suppressing complexity in the configuration of the medical observation device 10, leading to achievement of improve tissue visibility by the user. An example of the intraocular birefringence tissue is a retinal nerve fiber. In glaucoma, it is known that retinal nerve fibers are destroyed by intraocular pressure, and acquisition of interference patterns in a plurality of polarization directions as in the present embodiment makes it possible to improve the accuracy of diagnosis on the lesion.

Since other configurations, operations, and effects may be similar to those in the above-described embodiment, detailed description will be omitted here.

### 11. Eleventh Embodiment

Next, a medical observation device and an information processing device according to an eleventh embodiment of the present disclosure will be described in detail with reference to the drawings. In the following description, the configuration, operation, and effects similar to those of the above-described embodiments will be cited, thereby omitting redundant description. The following will describe an exemplary case where the medical observation device 6 described with reference to FIG. 10 in the sixth embodiment is used as a base. However, the configuration is not limited thereto, and it is also possible to use a medical observation device according to another embodiment or a modification thereof as a base.

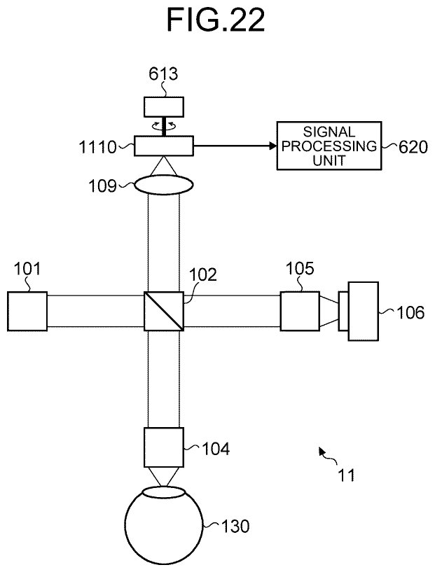

FIG. 22 is a schematic diagram illustrating a schematic configuration example of the medical observation device according to the present embodiment. As illustrated in FIG. 22, for example, a medical observation device 11 according to the present embodiment has a configuration similar to the medical observation device 6 described with reference to FIG. 10 in the sixth embodiment, but this configuration includes an event-based vision sensor (EVS) 1110 in place of the image sensor 110.

The EVS is an image sensor that detects event data and outputs a result of the detection. Specifically, when a change in luminance (or light intensity) is detected in a pixel, the EVS detects coordinates, directions (polarities), and time of the luminance change regarding the pixel (which are defined as event data) as an address event, and outputs the result of the detection (event data) synchronously or asynchronously.

A typical angiography examination acquires a plurality of two-dimensional OCT images or three-dimensional OCT images, and extracts images of blood vessels from information indicating difference among the images, thereby generating an angiographic image. In comparison, in the case of the EVS 1110, no signals are output from the pixel with no luminance change detected, while signals are output from the pixel with a luminance change due to activities such as a blood flow. Therefore, acquiring an en-face image using the EVS 1110 makes it possible to directly acquire an active image such as a blood vessel.

In addition, in general, the frame rate of EVS is about 1000 frame per second (fps), which is significantly higher than that of a normal image sensor. Therefore, the use of the EVS 1110 enables high-speed generation of an angiographic image.

Since other configurations, operations, and effects may be similar to those in the above-described embodiment, detailed description will be omitted here.

### 12. Hardware Configuration

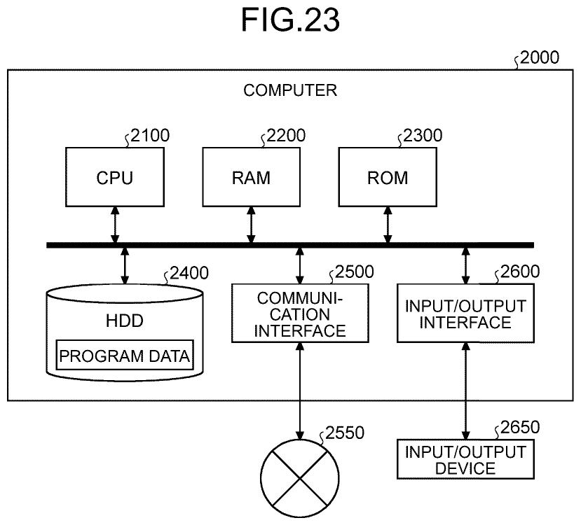

The signal processing units 120 and 620 according to the above-described embodiments and modifications thereof can be implemented by a computer 2000 having a configuration as illustrated in FIG. 23, for example. FIG. 23 is a hardware configuration diagram illustrating an example of the computer 2000 that implements functions of the signal processing units 120 and 620. The computer 2000 includes a CPU 2100, RAM 2200, read only memory (ROM) 2300, a hard disk drive (HDD) 2400, a communication interface 2500, and an input/output interface 2600. Individual components of the computer 2000 are interconnected by a bus 2050.

The CPU 2100 operates based on a program stored in the ROM 2300 or the HDD 2400 so as to control each of components. For example, the CPU 2100 develops the program stored in the ROM 2300 or the HDD 2400 into the RAM 2200 and executes processing corresponding to various programs.

The ROM 2300 stores a boot program such as a basic input output system (BIOS) executed by the CPU 2100 when the computer 2000 starts up, a program dependent on hardware of the computer 2000, or the like.

The HDD 2400 is a non-transitory computer-readable recording medium that records a program executed by the CPU 2100, data used by the program, or the like. Specifically, the HDD 2400 is a recording medium that records a program for executing individual operations according to the present disclosure, which is an example of program data 2450.

The communication interface 2500 is an interface for connecting the computer 2000 to an external network 2550 (for example, the Internet). For example, the CPU 2100 receives data from other devices or transmits data generated by the CPU 2100 to other devices via the communication interface 2500.

The input/output interface 2600 is an interface for connecting between an input/output device 2650 and the computer 2000. For example, the CPU 2100 receives data from an input device such as a keyboard or a mouse via the input/output interface 2600. In addition, the CPU 2100 transmits data to an output device such as a display, a speaker, or a printer via the input/output interface 2600. Furthermore, the input/output interface 2600 may function as a media interface for reading a program or the like recorded on predetermined recording media. Examples of the media include optical recording media such as a digital versatile disc (DVD) or a phase change rewritable disk (PD), a magneto-optical recording medium such as a magneto-optical disk (MO), a tape medium, a magnetic recording medium, and semiconductor memory.

For example, when the computer 2000 functions as the signal processing units 120 and 620 according to the above-described embodiment, the CPU 2100 of the computer 2000 executes the program loaded on the RAM 2200 to implement the functions of the signal processing units 120 and 620. In addition, the HDD 2400 stores programs according to the present disclosure, and the like. While the CPU 2100 executes program data 2450 read from the HDD 2400, the CPU 2100 may acquire these programs from another device via the external network 2550, as another example.

It should be noted that the embodiments and modifications disclosed herein are merely illustrative in all respects and are not to be construed as limiting. The above-described embodiments and modifications can be omitted, replaced, and changed in various forms without departing from the scope and spirit of the appended claims. For example, the above-described embodiments and modifications may be combined in whole or in part, and embodiments other than the above-described embodiments and modifications may be combined with the above-described embodiments or modifications. Furthermore, the effects of the present disclosure described in the present specification are merely illustrative, and other effects may be provided.

A technical category embodying the above technical idea is not limited. For example, the above-described technical idea may be embodied by a computer program for causing a computer to execute one or a plurality of procedures (steps) included in a method of manufacturing or using the above-described device. In addition, the above-described technical idea may be embodied by a computer-readable non-transitory recording medium in which such a computer program is recorded.

Note that the present technique can also have the following configurations.

(1)

A medical observation device comprising:
a light source that emits at least spatially incoherent light;
an image sensor that acquires an image of the light emitted from the light source and reflected by a subject; and
a signal processing unit that corrects a signal level obtained from first image data acquired by the image sensor, based on a wavefront aberration of the light reflected by the subject.
   (2)

The medical observation device according to (1), further comprising:
a reflection mirror movable along an optical axis of the light emitted from the light source; and
a beam splitter that splits the light emitted from the light source into first light incident on the subject and second light incident on the reflection mirror, and multiplexes the first light reflected by the subject and the second light reflected by the reflection mirror, wherein
the image sensor acquires an interference pattern created by the first light and the second light, as the first image data.
   (3)

The medical observation device according to (2), further comprising
a vibration mechanism that moves the reflection mirror along the optical axis.
   (4)

The medical observation device according to (3), wherein
the signal processing unit calculates the signal level based on an amplitude of a signal strength at each pixel obtained from two or more pieces of the first image data acquired by the image sensor during movement of the reflection mirror along the optical axis, and corrects the calculated signal level based on the wavefront aberration.
   (5)

The medical observation device according to any one of (1) to (4), further comprising
a wavefront sensor that detects a wavefront of the light reflected by the subject, wherein
the signal processing unit corrects the signal level based on the wavefront detected by the wavefront sensor.
   (6)

The medical observation device according to any one of (1) to (5), wherein
the signal processing unit corrects the signal level based on a region with less texture specified based on second image data of the subject acquired by the image sensor.
   (7)

The medical observation device according to any one of (1) to (6), further comprising
a stage on which the light source and the image sensor are fixed and which is movable with respect to the subject.
   (8)

The medical observation device according to any one of (1) to (6), further comprising:
an objective lens that irradiates the subject with the light emitted from the light source; and
a moving mechanism that moves the objective lens along an optical axis of the light emitted from the light source.
   (9)

The medical observation device according to any one of (1) to (8), further comprising
an adjustment mechanism that adjusts a rotation angle of the image sensor with respect to the image of the light emitted from the light source and reflected by the subject, with an optical axis of the light defined as a rotation axis.
   (10)

The medical observation device according to (9), wherein
the adjustment mechanism rotates the image sensor, with the optical axis of the light emitted from the light source defined as the rotation axis.
   (11)

The medical observation device according to (9), wherein
the adjustment mechanism rotates the image of the light reflected by the subject and incident on the image sensor.
   (12)

The medical observation device according to any one of (9) to (11), wherein
the image sensor includes a pixel array unit having a plurality of pixels two-dimensionally arranged in a matrix, and generates the first image data by driving a rectangular region, which is a part of the pixel array unit and is long in one direction.
   (13)

The medical observation device according to any one of (9) to (12), wherein
the image sensor includes a pixel having light receiving sensitivity to light having a wavelength other than a visible light spectrum.
   (14)

The medical observation device according to any one of (9) to (12), wherein
the image sensor includes two or more pixels having light receiving sensitivity to light polarized in mutually different directions.
   (15)

The medical observation device according to any one of (9) to (12), wherein
the image sensor is an event-based vision sensor (EVS) that outputs event data indicating a pixel having a luminance change detected.
   (16)

The medical observation device according to any one of (1) to (15), the device being either a surgical microscope or a funduscope.

(17)

An information processing device comprising a signal processing unit that corrects a signal level obtained from image data of an image of light emitted from a light source that emits at least spatially incoherent light and reflected by a subject, based on a wavefront aberration of the light reflected by the subject.

(18)

A medical observation device including:
a light source that emits light;
an image sensor that acquires an image of the light emitted from the light source and reflected by a subject; and
an adjustment mechanism that adjusts a rotation angle of the image sensor with respect to the image of the light emitted from the light source and reflected by the subject, with an optical axis of the light defined as a rotation axis.
   (19)

The medical observation device according to (18), further including:
a reflection mirror movable along the optical axis of the light emitted from the light source; and
a beam splitter that splits the light emitted from the light source into first light incident on the subject and second light incident on the reflection mirror, and multiplexes the first light reflected by the subject and the second light reflected by the reflection mirror, in which the image sensor acquires an interference pattern created by the first light and the second light, as image data.
   (20)

The medical observation device according to (19), further including
a vibration mechanism that moves the reflection mirror along the optical axis.
   (21)

The medical observation device according to (19) or (20), in which
the image sensor includes a pixel array unit having a plurality of pixels two-dimensionally arranged in a matrix, and generates the image data by driving a rectangular region, which is a part of the pixel array unit and is long in one direction.
   (22)

The medical observation device according to any one of (18) to (21), in which
the adjustment mechanism rotates the image sensor, with the optical axis of the light emitted from the light source defined as the rotation axis.
   (23)

The medical observation device according to any one of (18) to (21), in which
the adjustment mechanism rotates the image of the light reflected by the subject and incident on the image sensor.
   (24)

The medical observation device according to any one of (18) to (23), in which
the image sensor includes a pixel having light receiving sensitivity to light having a wavelength other than a visible light spectrum.
   (25)

The medical observation device according to any one of (18) to (23), in which
the image sensor includes two or more pixels having light receiving sensitivity to light polarized in mutually different directions.
   (26)

The medical observation device according to any one of (18) to (23), in which
the image sensor is an event-based vision sensor (EVS) that outputs event data indicating a pixel having a luminance change detected.
   (27)

The medical observation device according to any one of (18) to (26), the device being either a surgical microscope or a funduscope.

### REFERENCE SIGNS LIST

1, 2, 3, 4, 6, 6A, 7, 7A, 9, 10, 11 MEDICAL OBSERVATION DEVICE
5 OBSERVATION DEVICE
21 to 24 PIXEL
101 INCOHERENT LIGHT SOURCE
102, 103 BEAM SPLITTER
104, 105 OBJECTIVE LENS
106 VIBRATION MECHANISM
107, 109 IMAGING LENS
108 WAVEFRONT SENSOR
110, 910 IMAGE SENSOR
111 PIXEL ARRAY UNIT
112, 112a DRIVE AREA
120, 220, 620 SIGNAL PROCESSING UNIT
121, 221 CORRECTION UNIT
122, 222 CORRECTION AMOUNT CALCULATION UNIT
130, 530 SUBJECT
301 STAGE
414, 415 MOVING MECHANISM
601 COHERENT LIGHT SOURCE
613 ROTATION MECHANISM
720 IMAGE ROTATOR
721 to 723 MIRROR
724 DOVE PRISM
1010 POLARIZATION IMAGE SENSOR
1110 EVS

1. A medical observation device comprising:
a light source that emits at least spatially incoherent light;
an image sensor that acquires an image of the light emitted from the light source and reflected by a subject; and
a signal processing unit that corrects a signal level obtained from first image data acquired by the image sensor, based on a wavefront aberration of the light reflected by the subject.

2. The medical observation device according to claim 1, further comprising:
a reflection mirror movable along an optical axis of the light emitted from the light source; and
a beam splitter that splits the light emitted from the light source into first light incident on the subject and second light incident on the reflection mirror, and multiplexes the first light reflected by the subject and the second light reflected by the reflection mirror, wherein
the image sensor acquires an interference pattern created by the first light and the second light, as the first image data.

3. The medical observation device according to claim 2, further comprising
a vibration mechanism that moves the reflection mirror along the optical axis.

4. The medical observation device according to claim 3, wherein
the signal processing unit calculates the signal level based on an amplitude of a signal strength at each pixel obtained from two or more pieces of the first image data acquired by the image sensor during movement of the reflection mirror along the optical axis, and corrects the calculated signal level based on the wavefront aberration.

5. The medical observation device according to claim 1, further comprising
a wavefront sensor that detects a wavefront of the light reflected by the subject, wherein
the signal processing unit corrects the signal level based on the wavefront detected by the wavefront sensor.

6. The medical observation device according to claim 1, wherein
the signal processing unit corrects the signal level based on a region with less texture specified based on second image data of the subject acquired by the image sensor.

7. The medical observation device according to claim 1, further comprising
a stage on which the light source and the image sensor are fixed and which is movable with respect to the subject.

8. The medical observation device according to claim 1, further comprising:
an objective lens that irradiates the subject with the light emitted from the light source; and
a moving mechanism that moves the objective lens along an optical axis of the light emitted from the light source.

9. The medical observation device according to claim 1, further comprising
an adjustment mechanism that adjusts a rotation angle of the image sensor with respect to the image of the light emitted from the light source and reflected by the subject, with an optical axis of the light defined as a rotation axis.

10. The medical observation device according to claim 9, wherein
the adjustment mechanism rotates the image sensor, with the optical axis of the light emitted from the light source defined as the rotation axis.

11. The medical observation device according to claim 9, wherein
the adjustment mechanism rotates the image of the light reflected by the subject and incident on the image sensor.

12. The medical observation device according to claim 9, wherein
the image sensor includes a pixel array unit having a plurality of pixels two-dimensionally arranged in a matrix, and generates the first image data by driving a rectangular region, which is a part of the pixel array unit and is long in one direction.

13. The medical observation device according to claim 9, wherein
the image sensor includes a pixel having light receiving sensitivity to light having a wavelength other than a visible light spectrum.

14. The medical observation device according to claim 9, wherein
the image sensor includes two or more pixels having light receiving sensitivity to light polarized in mutually different directions.

15. The medical observation device according to claim 9, wherein
the image sensor is an event-based vision sensor (EVS) that outputs event data indicating a pixel having a luminance change detected.

16. The medical observation device according to claim 1, the device being either a surgical microscope or a funduscope.

17. An information processing device comprising a signal processing unit that corrects a signal level obtained from image data of an image of light emitted from a light source that emits at least spatially incoherent light and reflected by a subject, based on a wavefront aberration of the light reflected by the subject.
